(19) United States
(12) Patent Application Publication
(10) Pub. No.: US 2011/0125155 A1
(43) Pub. Date: May 26, 2011

(54) HUMERAL HEAD SHAPING DEVICE AND METHOD

(75) Inventors: Austin W. Mutchler, Warsaw, IN (US); Matthew J. Purdy, Eden Prairie, MN (US); Robert Courtney, JR., Pierceton, IN (US)

(73) Assignee: TORNIER, INC., Edina, MN (US)

(21) Appl. No.: 12/953,895

(22) Filed: Nov. 24, 2010

Related U.S. Application Data
(60) Provisional application No. 61/264,042, filed on Nov. 24, 2009.

(51) Int. Cl. A61B 17/56

(52) U.S. Cl. 606/87

(57) ABSTRACT

Provided is a method and device for shaping a greater tuberosity of a humerus in preparing for receiving a prosthetic humeral head. The device shaves bone from the greater tuberosity such that the greater tuberosity is not resected or completely removed.

### CROSS REFERENCE TO RELATED APPLICATIONS

This application claims priority to U.S. Provisional Application No. 61/264,042, filed Nov. 24, 2009 and entitled, “TUBEROSITY MILL-EH2 SYSTEM,” the entire contents of which are incorporated herein by reference.

### TECHNICAL FIELD

The present invention is related generally to the field of prosthetic humeral heads. In particular, the present invention is a device and method of preparing a humerus for implantation of a prosthetic humeral head.

### BACKGROUND

Generally, current methodologies of preparing the greater tuberosity of a humerus for implantation of a prosthetic humeral head do not address cuff tear arthropathy (CTA). One device for providing a humeral head while addressing CTA involves a cutting block that attaches to the broach. The block is used with a saw blade to remove the greater tuberosity in its entirety.

### SUMMARY

Some embodiments relate to methods and devices for shaping a greater tuberosity of a humerus in preparing for receiving a prosthetic humeral head. The device shaves bone from the greater tuberosity such that the greater tuberosity is not resected or completely removed.

Other embodiments relate to a device for shaping the greater tuberosity of a humerus, the device including a shaft having a first end, a second end, a proximal portion, and a distal portion, the distal portion having a longitudinal axis. The device also includes a guide positioned between the first end and the second end of the shaft, the guide having a spoke defining a cutting face, the spoke extending outward and downward from the shaft, the shape of the cutting face configured to produce a desired shape of the greater tuberosity.

Still other embodiments relate to a method for shaping the greater tuberosity of a humerus, the method including inserting the distal portion of a shaping device into a humeral shaft of a humerus until a cutting face of a spoke contacts a greater tuberosity of the humerus and rotating the shaping device around the longitudinal axis of the distal portion with a downward force into the humerus such that the greater tuberosity is formed with a desired shape.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1A is a side view of a first embodiment of a device for shaping a greater tuberosity of a humerus.

FIG. 1B is a perspective view of the device of FIG. 1A.

FIGS. 2 to 5 are schematic views of steps for shaping a greater tuberosity of a humerus, according to some embodiments.

FIGS. 6A to 6C are schematic views of steps for shaping a greater tuberosity of a humerus with a second embodiment of a device for shaping a greater tuberosity of a humerus using a lateral approach, according to some embodiments.

FIGS. 7A to 7C are schematic views of steps for shaping a greater tuberosity of a humerus with the second embodiment of the device for shaping a greater tuberosity of a humerus using an axial approach, according to some embodiments.

FIGS. 8A and 8B are side views of a third embodiment of a device for shaping a greater tuberosity of a humerus.

FIGS. 8C and 8D are perspective views of the device of FIG. 8A.

FIG. 9 is a side view of a shaped greater tuberosity of the humeral head after shaping, according to some embodiments.

FIG. 10 shows a side view of a humerus with a prosthetic head installed, according to some embodiments.

FIG. 11 shows a closer view of the spoke and cutting face shown in FIG. 5.

### DETAILED DESCRIPTION

Various embodiments relate to a device for shaping a greater tuberosity of a humerus in preparing for implantation of a prosthetic humeral head, as well as methods of preparing the greater tuberosity of the humerus without resection thereof. For example, in some embodiments, the device provides the ability to smooth or shape a greater tuberosity of a humerus to fit the underside of a cuff tear arthropathy (CTA) humeral head without resecting the greater tuberosity. While some features and advantages of various embodiments have been provided above by way of example, various additional or alternate features and advantages are contemplated. For example, while various embodiments are described in association with shaping of the greater tuberosity of a humerus, in other embodiments devices and methods within the scope of invention are utilized to shape other portions of the anatomy, such as the head of a femur, for example.

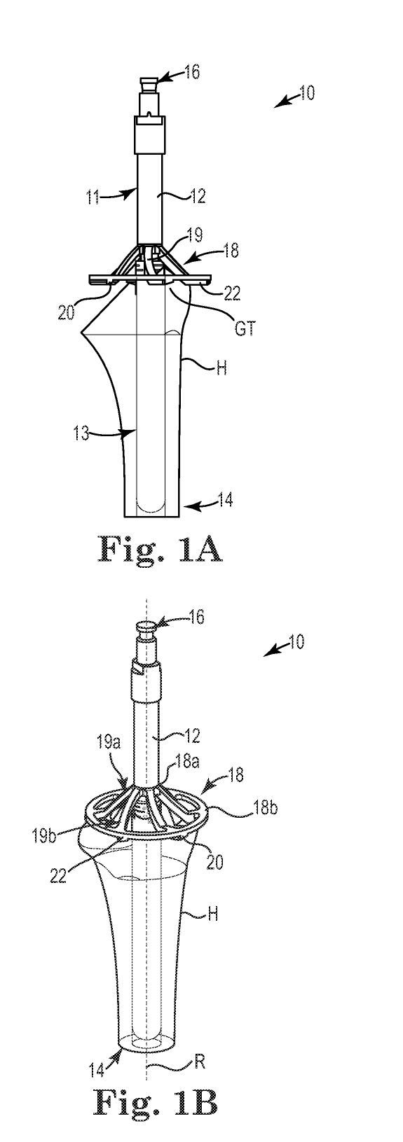

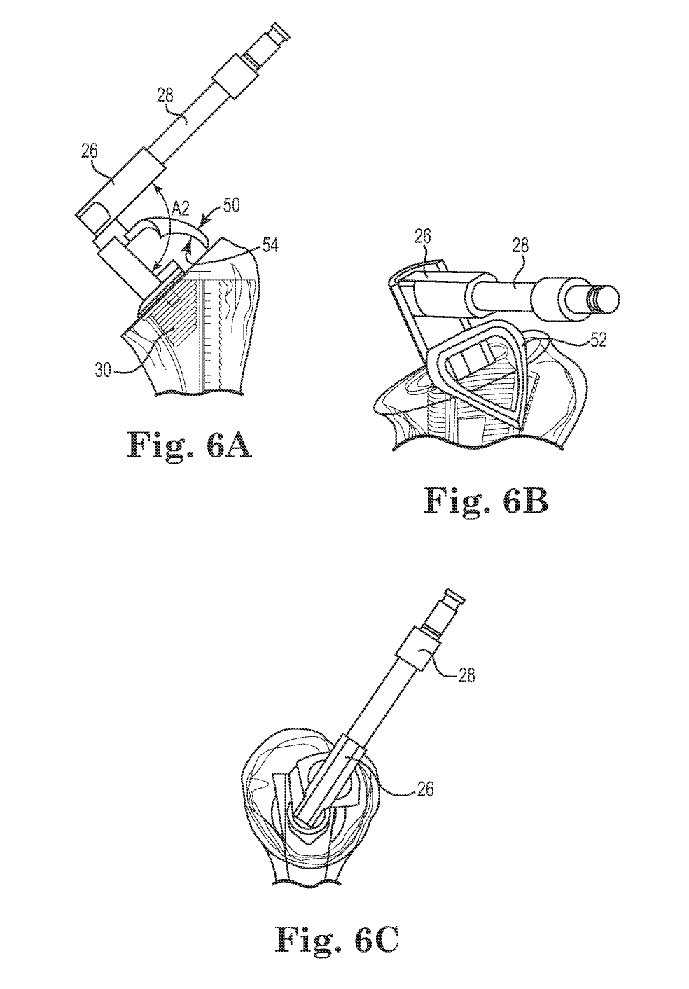

FIG. 1A and FIG. 1B are side and perspective views, respectively, of a first embodiment of a device for shaping a greater tuberosity of a humerus H. The tuberosity mill 10 shown in FIGS. 1A and 1B includes a shaft 12 having a first end 14 and a second end 16, a guide 18 positioned between the first end 14 and the second end 16 of the shaft 12 that, in some embodiments, attaches to a broach or trial 30 (FIG. 6).

In some embodiments, the shaft 12 includes a proximal portion 11 extending from the second end 16 to the guide 18 and a distal portion 13 extending from the guide 18 to the first end 14, the distal portion 13 having a longitudinal axis of rotation R. In one embodiment, the distal portion 13 of the shaft 12 is smooth and generally cylindrical to facilitate insertion into the humerus H. In one embodiment, a standard T-handle (not shown) is attached to the second end 16 of the shaft 12 of the tuberosity mill 10.

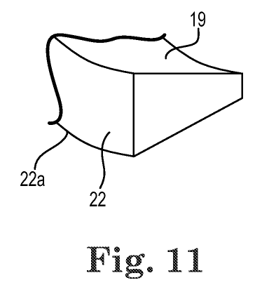

In some embodiments, the guide 18 includes a plurality of spokes 19 each having a proximal portion 19a and distal portion 19b. Each of the spokes 19 forms a cutting face 22 (FIG. 11) have a cutting edge 22a (also defined herein as a tooth) to form a cutter 20, such that the cutter 20 includes a plurality of cutting edges 22a. In some embodiments, the neck portion 18a is shaped as a ring that is adapted to be coaxially received over one or more portions of the shaft 12.

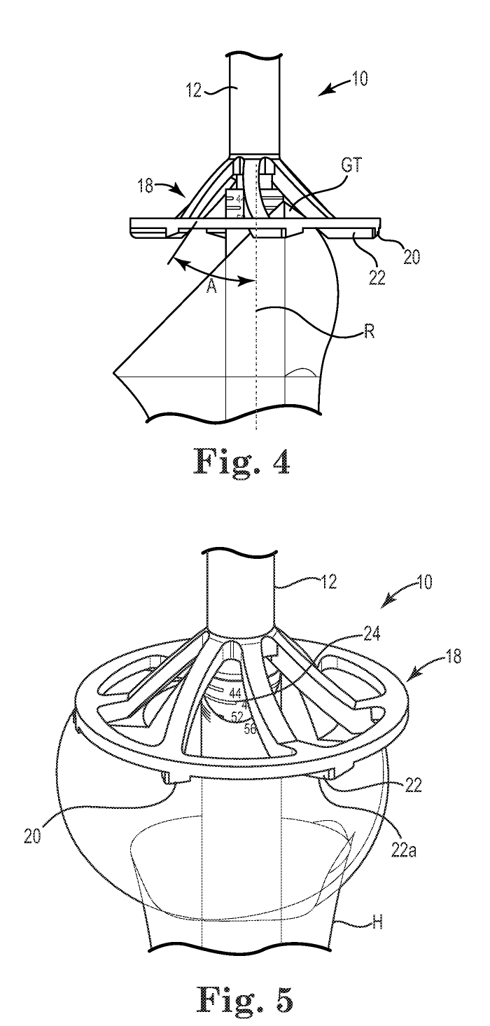

In some embodiments, as shown in FIG. 5, the spokes 19 traverse a substantially twisted, arcuate path between the neck portion 18a and the base portion 18b to define the cutting faces 22, although other configurations are contemplated. The spokes 19 extend radially outward and downward from a neck portion 18a to a base portion 18b. As shown, the proximal portion 19a defines an angle A (FIG. 4) of less than 90 degrees with an axis of rotation R of the guide 18 and the distal portion defines an angle of about 90 degrees with the axis of rotation R of the guide 18. In different terms, the spokes 19 optionally splay outwardly at the distal portions 19b, such that the distal portions 19b each of the spokes 19 are oriented substantially radially, or orthogonally, to the axis of rotation R of the cutter 20 and the longitudinal axis of the distal portion 13 of the shaft 12.

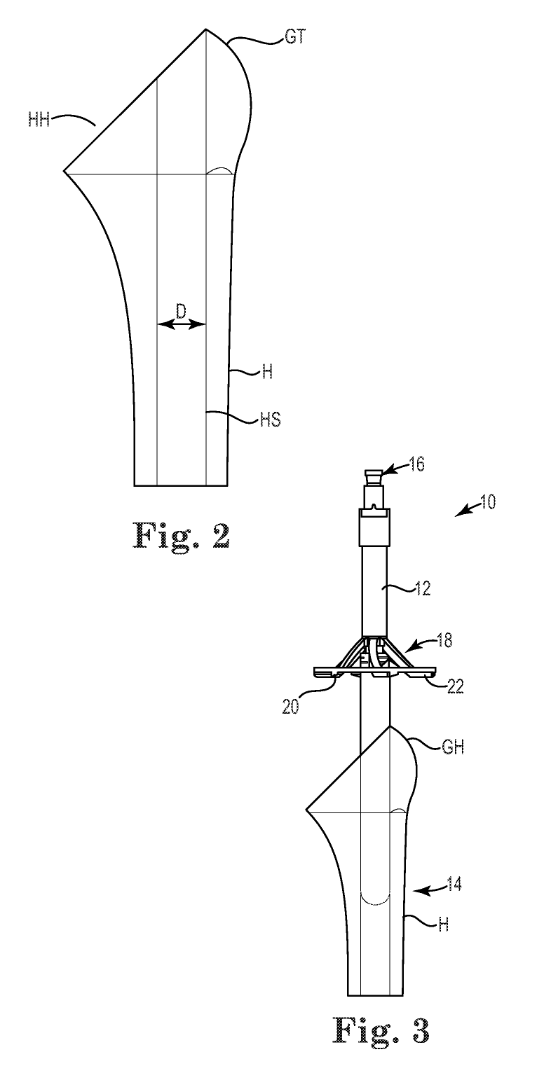

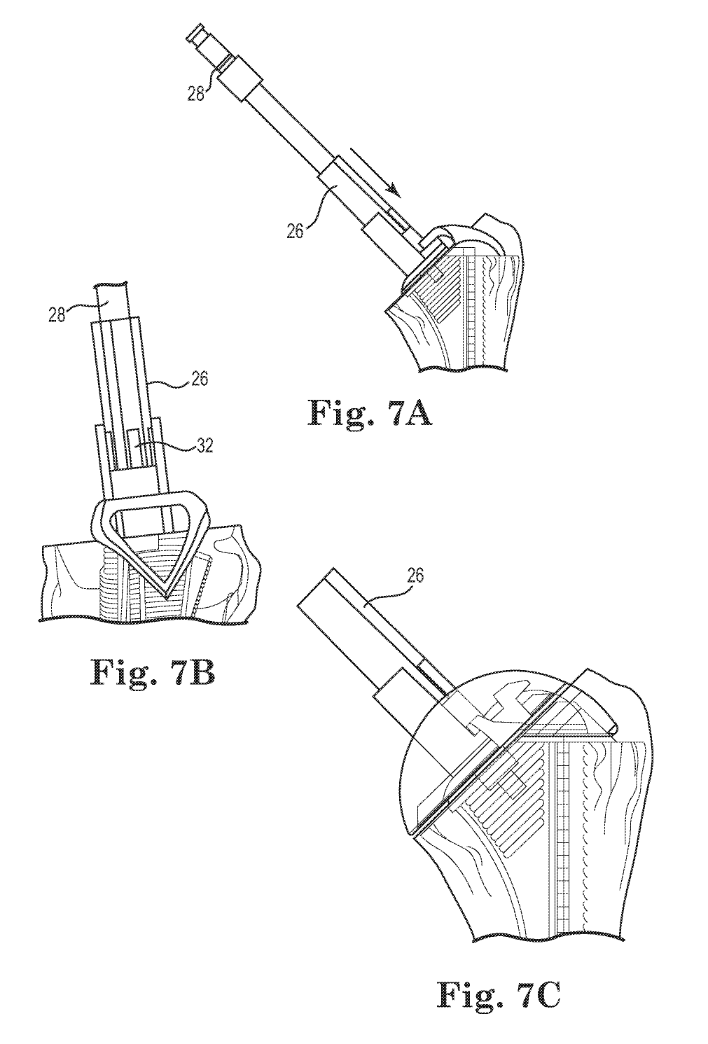

Generally, the proximal portions 19a of each of the spokes 19 are interconnected and supported by the neck portion 18a (which, in turn, is connected to the shaft 12) and the distal portions 19b of each of the spokes 19 are interconnected and supported by the base portion 18b. In general terms, the cutter 20 is secured to the shaft such that the spokes 19 are rotated upon rotating the shaft 12 about the axis of rotation R. While FIGS. 1-5 generally show the distal portions 19b of the spokes 19 being interconnected or attached to one another indirectly by the base portion 18b, in other embodiments the distal portions of multiple spokes are attached directly to one another as shown in FIGS. 6A-6C and 7A-7C, for example.

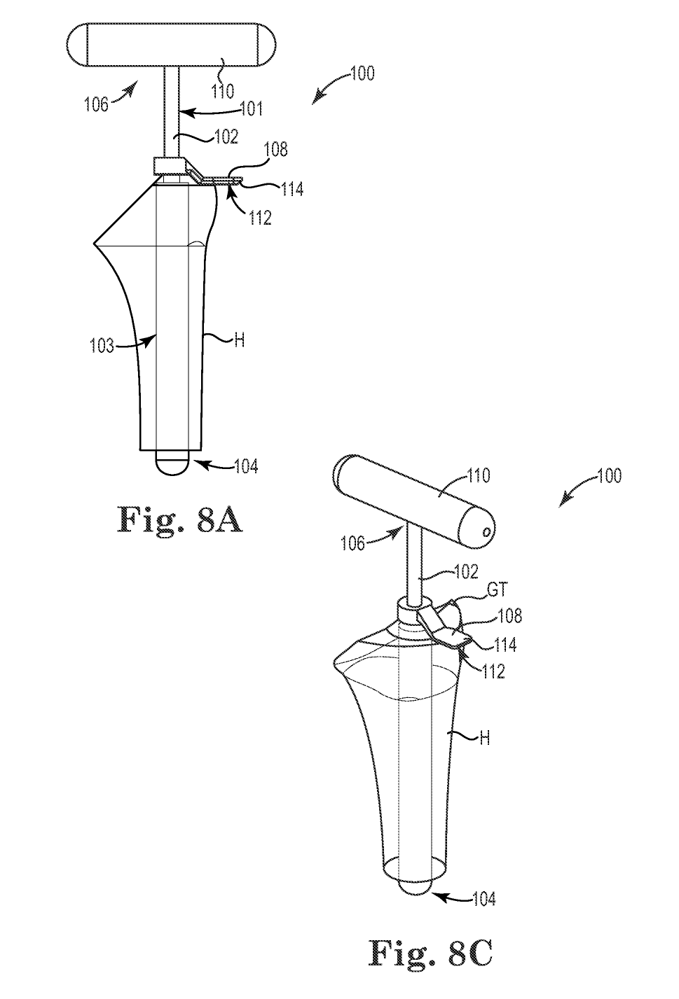

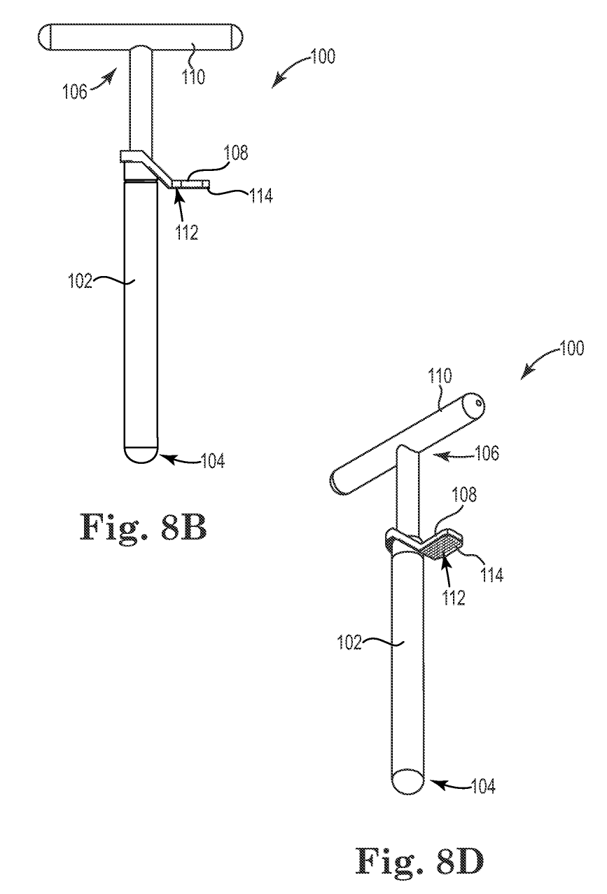

As referenced above, in some embodiments, the cutting edges 22a of the spokes 19 are sharp and adapted for shaping the greater tuberosity GT of the proximal humerus H. For example, as shown in FIG. 5, each of the cutting edges 22a is angled, such that the spokes 19 are adapted to shave bone in order to produce a desired shape of the greater tuberosity GT without resecting the greater tuberosity GT. As shown, the spokes 19 are configured to produce the desired shape (e.g., the cutting edges 22a having a desired profile for generating the desired shape for the greater tuberosity GT, such as the angled, then relatively horizontal profile shown in FIGS. 1A and 1B). In other embodiments, the cutter 20 includes a single spoke 19 forming a single cutting edge 22a that is configured to produce the desired shape of the greater tuberosity GT (e.g., similar to embodiments described in association with FIGS. 8A to 8D).

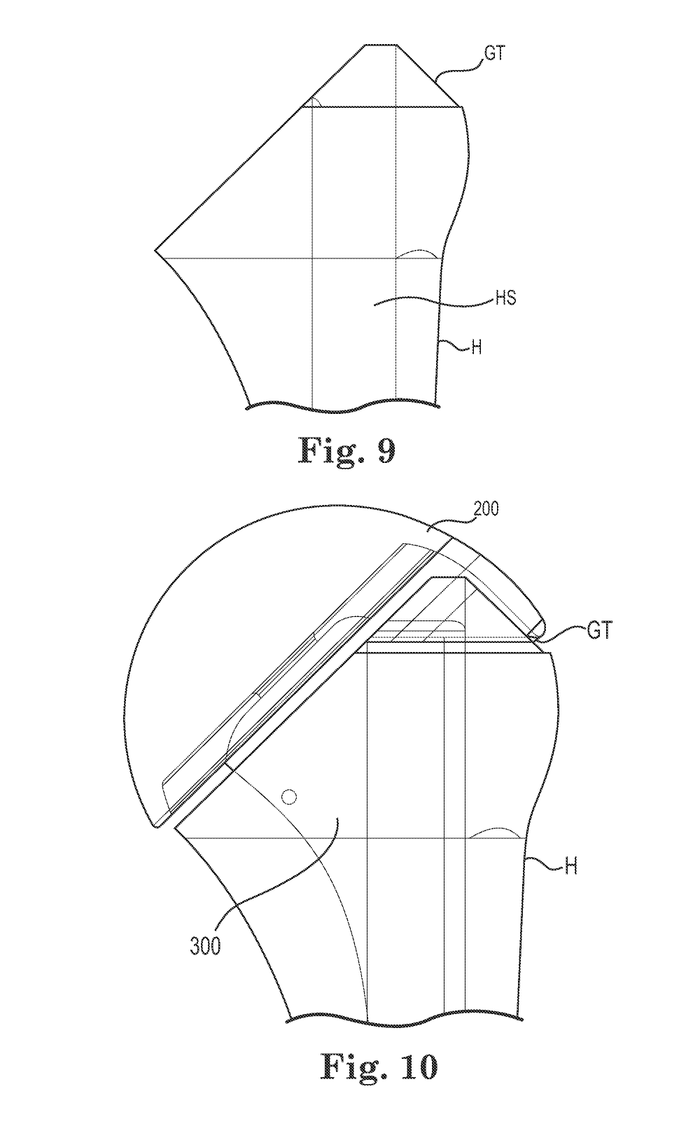

The desired shape of the greater tuberosity GT of the humerus H, and thus the configuration of the guide 18, depends, among other things, on the prosthetic humeral head to be used. In some embodiments, the guide 18 is configured to form, without limitation, generally rounded, triangular, and/or quadrilateral shapes, including, for example, the cone shape having a generally equilateral trapezoid cross-section shown in FIG. 9. In some embodiments, the guide 18 is removable and provided as one of a plurality of guides having different configurations, such that the mill 10 is able to be fitted with guides having cutters of different sizes and/or shapes.

As indicated in FIG. 5, the tuberosity mill 10 optionally includes a plurality of markings 24 (FIG. 5) along a length of the shaft 12. In some embodiments, the markings 24 are calibrated to indicate a depth to which the distal portion 13 of the shaft 12 is inserted in the humeral shaft HS when the greater tuberosity GT is milled to the desired shape. If desired, the markings 24 are calibrated to provide an indication that the greater tuberosity GT is shaped to fit a selected prosthetic humeral head. While numerical markings are shown, graphics, color coding, other indicia, and combinations thereof are also contemplated.

Some methods of shaping the greater tuberosity GT of the proximal humerus H with the tuberosity mill 10 are illustrated in FIGS. 3 to 5. In some embodiments, as a first step in shaping the greater tuberosity GT, the humeral head HH is removed from the humerus H and the humerus H is reamed to diameter D. In other embodiments, the humeral head HH is removed following reaming of the humerus H and/or shaping of the greater tuberosity GT.

In a second step shown in FIG. 3, the first end 14 of the shaft 12 of the tuberosity mill 10 is inserted into the humeral shaft HS of the humerus H. In one embodiment, the distal portion 13 of shaft 12 of the tuberosity mill 10 is inserted using the T-handle or power driver (not shown).

In a third step shown in FIG. 4, the tuberosity mill 10 is inserted into the humerus H until the cutter 20 of the tuberosity mill 10 contacts the greater tuberosity GT. When the cutter 20 of the tuberosity mill 10 is in contact with the humerus H, the guide 18 is rotated by rotating the tuberosity mill 10 with a downward force into the humerus H. As the tuberosity mill 10 is pushed into the humerus H, the spokes 19 of the cutter 20 shave the bone from the humerus H. In some embodiments, the spokes 19 of the tuberosity mill 10 are designed to shave thin layers of bone until the desired shape is achieved.

In a fourth step shown in FIG. 5, the tuberosity mill 10 continues to be advanced into the humerus H until the desired shape is achieved. For example, in some embodiments, the tuberosity mill 10 is advanced until the markings 22 along the length of the shaft 12 of the tuberosity mill 10 reflect that a desired greater tuberosity shape has been reached.

In some embodiments, an angle A2 (FIG. 6A) between the proximal portion 11 and the distal portion 13 of the shaft 12 is adjustable. For example, as shown in FIGS. 6A-6C and 7A-7C, the cutters 20 of the tuberosity mill 10 are modular and are optionally utilized with drivers and/or other components adapted for axial arthroplasty approaches (also described herein as a standard approach) and/or laterally for a lateral arthroplasty approaches to the humerus H. In some embodiments, the tuberosity mill 10 includes a joint 32 in the proximal portion 11 of the shaft 12 such that the proximal portion 11 can be angled for lateral approach. The joint 32 can be locked using, for example a sleeve 26, at the appropriate angle for use in an axial approach.

FIGS. 6A-6C show schematic diagrams of a lateral approach for shaping the greater tuberosity GT with another cutter 50 for shaping a greater tuberosity of a humerus, according to some embodiments. As shown, the cutter 50 includes two spokes 52 converging distally with one another and defining curved cutting faces 54 for a cutting operation. Though the cutter 50 is shown in FIGS. 6A to 6C and 7A to 7C, it should be understood that a similar axial and/or lateral procedures are optionally accomplished with other embodiment cutters described herein, such as cutter 20.

FIG. 6A is a schematic view of a first step in the lateral approach, FIG. 6B is a schematic view of a second step in the lateral approach and FIG. 6C is a schematic view of a third step in the lateral approach. As can be seen in FIG. 6A, in the lateral approach, a sleeve 26 of the proximal shaft portion 28 locks the proximal shaft portion 28 in lateral alignment with the cutter 20 of the tuberosity mill 10. FIG. 6B illustrates the cutter 50 being captured in the guide 18. In FIG. 6C, the cutter 50 is rotated from side to side to remove bone.

FIGS. 7A-7C show schematic diagrams of an axial approach for shaping the greater tuberosity GT, according to some embodiments. FIG. 7A is a schematic view of a first step in the axial approach, FIG. 7B is a schematic view of a second step in the axial approach and FIG. 7C is a third step in the axial approach. The axial approach is similar to the lateral approach except that the proximal shaft portion 28 is locked in axial alignment with the cutter 50 of the tuberosity mill 10.

FIGS. 8A and 8B are side views and FIGS. 8C and 8D are perspective views of a third embodiment of a device for shaping the greater tuberosity GT of the humerus H. As shown in FIGS. 8A to 8D, the bone rasp 100 shown is used to shape the greater tuberosity GT for receiving a prosthetic humeral head. The bone rasp 100 generally includes a shaft 102 having a first end 104 and a second end 106, a protruding rasp 108, also described as a spoke, positioned between the first end 104 and the second end 106, and a handle 110 attached the second end 106. The shaft 102 includes a proximal portion 101 extending from the second end 106 to the protruding rasp 108 and a distal portion 103 extending from the protruding rasp 108 to the first end 104. In one embodiment, the distal portion 103 of the shaft 102 is smooth and generally cylindrical to facilitate insertion into the humerus H. In some embodiments, the shaft 102 includes one or more of the features described herein for the shaft 12 of the disclosed mill 10.

The protruding rasp 108 defines one or more cutting edges for removing bone. For example, in some embodiments, the protruding rasp 108 has a bottom surface 112 forming a plurality of sharp teeth 114, or cutting edges, for shaving bone, similar to the cutting edges 22a of the cutter 20. In some embodiments, the protruding rasp 108 is removably attached to the shaft 102 and exchangeable with differently configured protruding rasps (not shown) for forming the greater tuberosity.

In use, the first end 104 of the shaft 102 is inserted into the humeral shaft HS of the humerus H until the rasp 108 contacts the greater tuberosity GT. The bone rasp 100 is then rotated using the handle 110. In one embodiment, the handle 110 is rotated about 90 degrees anterior and about 90 degrees posterior while applying force on the handle 110 in the direction of the humeral shaft HS. As the bone rasp 100 is rotated, the teeth 114 on the bottom surface 112 of the rasp 108 shave away layers of the greater tuberosity GT. The bone rasp 100 is rotated until the desired amount of the greater tuberosity GT has been shaved off. According to some embodiments, using the bone rasp 100 helps allow the greater tuberosity GT to be shaped to accommodate the prosthetic humeral head, rather than resecting or completely removing the greater tuberosity GT.

Once the greater tuberosity GT has been properly shaped, the particular shaping device (e.g., devices 10 or 100 referenced in the foregoing) is removed from the humerus H and the prosthetic humeral head is installed. For example, FIG. 9 shows the shaved greater tuberosity GT of the humeral head HH after milling, according to some embodiments, and FIG. 10 shows a side view of the humerus H with the prosthetic humeral head 200 installed over the greater tuberosity GT as shaped. In some embodiments, after shaping the greater tuberosity GT, broaching and/or trialing of the proximal humerus H is completed, where as shown in FIG. 10, the prosthetic humeral head 200 is then placed on a broach and/or trial component 300 with a complementary fit, the prosthetic humeral head 200 having a receptacle sized and shaped, or otherwise configured, to receive the shaped greater tuberosity GT in a complementary fit.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above described features.

What is claimed is:

1. A device for shaping the greater tuberosity of a humerus, comprising:
a shaft having a first end, a second end, a proximal portion, and a distal portion, the distal portion having a longitudinal axis; and
a guide positioned between the first end and the second end of the shaft, the guide having a spoke defining a cutting face, the spoke extending outward and downward from the shaft, the shape of the cutting face configured to produce a desired shape of the greater tuberosity.

2. The device of claim 1, wherein the cutting face of the spoke forms an angle of less than 90 degrees with the longitudinal axis of the distal portion.

3. The device of claim 2, wherein the spoke defines a proximal portion and a distal portion, the proximal portion extending radially outward and downward and the distal portion splaying radially outward relative to the shaft.

4. The device of claim 1, wherein the cutting face of the spoke is curved.

5. The device of claim 1, wherein the device comprises a plurality of spokes.

6. The device of claim 5, wherein the proximal portions of the spokes are interconnected by a neck portion and the distal portions of the spokes are interconnected by a base portion.

7. The device of claim 6, wherein the spokes traverse a substantially twisted, arcuate path between the neck portion and the base portion.

8. The device of claim 5, wherein the distal portions of the spokes are directly attached to one another.

9. The device of claim 1, wherein the spoke comprises a plurality of cutting edges.

10. The device of claim 1, wherein the desired shape is a cone shape having a generally equilateral trapezoid cross-section.

11. The device of claim 1, further comprising a plurality of calibrated markings along a length of the shaft.

12. The device of claim 11, wherein the markings correspond to a desired greater tuberosity shape.

13. The device of claim 1, further comprising a joint in the proximal portion.

14. The device of claim 1, further comprising a broach connected to the shaft.

15. A method for shaping the greater tuberosity of a humerus, the method comprising:
inserting a distal portion of a shaping device into a humeral shaft of the humerus until a cutting face of the spoke contacts the greater tuberosity of the humerus, the shaping device having shaft and a guide secured to the shaft and including a spoke defining a cutting face, the spoke extending outward and downward from the shaft, the shape of the cutting face configured to produce a desired shape of the greater tuberosity; and
rotating the shaping device around a longitudinal axis of the shaft with a downward force into the humerus such that the greater tuberosity is formed with a desired shape.

16. The method of claim 15, wherein the device is advanced until markings along the length of the shaft of the device reflect that the desired greater tuberosity shape has been achieved.

17. The method of claim 15, wherein the desired shape is cone shape having a generally equilateral trapezoid cross-section.

18. The method of claim 15, further comprising shaping the greater tuberosity using an axial approach.

19. The method of claim 15, further comprising shaping the greater tuberosity using a lateral approach.

20. The method of claim 15, further comprising positioning a prosthetic humeral head over the shaped greater tuberosity in a complementary fit.
